(19) United States
(12) Patent Application Publication
(10) Pub. No.: US 2006/0035859 A1
(43) Pub. Date: Feb. 16, 2006

(54) Treating severe and acute viral infections

(75) Inventors: William A. Carter, Spring City, PA (US); David Strayer, Bryn Mawr, PA (US)

Correspondence Address: NIXON & VANDERHYE, PC, 901 NORTH GLEBE ROAD, 11TH FLOOR, ARLINGTON, VA, 22203, US

(73) Assignee: HEMISPHERx BIOPHARMA, Philadelphia, PA (US)

(21) Appl. No.: 11/244,107

(22) Filed: Oct. 6, 2005

Related U.S. Application Data
(63) Continuation-in-part of application No. 10/842,474, filed on May 11, 2004.
(60) Provisional application No. 60/470,893, filed on May 16, 2003.
(60) Provisional application No. 60/517,882, filed on Nov. 7, 2003.

(51) Int. Cl. A61K 48/00; A61K 38/21

(52) U.S. Cl. 514/44; 424/85.7; 514/43

(57) ABSTRACT

Severe acute respiratory syndrome is treated with a natural human alpha interferon, a dsRNA or both natural human alpha interferon and a dsRNA. Avian influenza is treated with natural human alpha interferon, neuraminidase inhibitor(s) and ribavirin. Effects of influenza virus are mitigated with a dsRNA in combination with a neuraminidase influenza virus inhibitor. These two products, dsRNA, and alpha interferon, have therapeutic utility either given preventively (prophylactically) or in treatment of active disease. These unique immunological/antiviral actions, operating through immunological “cascades” ameliorates the lethal effects of viral mutation which, by causing resistance to commonly available drugs, greatly accelerates the death rate. For example, in 1918-1920, Avian Influenza caused the death of approximately 40 million people worldwide (ref. National Geographic, September 2005).

### CROSS REFERENCE TO RELATED APPLICATIONS

This application claims priority from U.S. provisional patent applications Ser. No. 60/470,893 filed May 16, 2003 and Ser. No. 60/517,882 filed Nov. 7, 2003 and is a continuation-in-part of application Ser. No. 10/842,474 filed May 11, 2004.

### BACKGROUND

Procedures are provided for combating the effects of coronavirus-induced conditions by the administration of an α-interferon composed of a mixture of naturally occurring α-interferons or a synthetic, specifically configured, double-stranded ribonucleic acid (dsRNA) or both an α-interferon and a dsRNA.

Severe Acute Respiratory Syndrome (SARS) is a new disease that is rapidly spreading within China and other-countries around the world. Although, a combination of ribavirin, a synthetic, non-interferon-inducing, broad spectrum antiviral nucleoside, and corticosteroids is commonly used as therapy, especially in China, laboratory testing by the National Institutes of Health (NIH) found ribavirin to have no effect on this coronavirus. This lack of efficacy suggests the need for an effective therapeutic regimen.

### BRIEF DESCRIPTION OF THE DRAWINGS

FIG. 1 is a graph showing percent of control of influenza virus infected cells by Ampligen® as in Example 3, and

FIG. 2 is a graph showing percent of control of influenza virus infected cells by Oseltamivir as in Example 3.

### DESCRIPTION OF THE INVENTION

Described is use of an α-interferon, preferably a natural, multi-species α-interferon in the treatment of the symptoms associated with SARS in patients including human patients infected with the SARS virus, also referred to as the SARS-associated coronavirus (SARS-CoV) or a susceptible viral infection other than SARS which has a common mechanism of viral multiplication or pathogenesis, in whole or in part, similar to that of SARS. Alternatively, a dsRNA may be used in the treatment of the symptoms associated with SARS-associated coronavirus in patents including human patients infected with the SARS-associated coronavirus. Also described is the coordinated use of both (1) an α-interferon, preferably a natural, multi-species α-interferon and conjointly therewith (2) a dsRNA in the treatment of the symptoms associated with SARS-associated coronavirus in patients including human patients infected the SARS-associated coronavirus. Procedures for attaining a favorable therapeutic and clinical result and compositions for accomplishing the same are described. Preferably the dsRNA is administered with the α-interferon and preferably the dsRNA is rIₙ·r(C₁₂U)_{n.}, Poly A·Poly U or rIₙ·r(C₂₉,G)ₙ, in which r is ribo.

In the context of the present invention, what is meant by “coordinated” use is, independently, either (i) co-administration, i.e. substantially simultaneous or sequential administration of the α-interferon and of the dsRNA, or (ii) the administration of a composition comprising the α-interferon and the dsRNA in combination and in a mixture, in addition to optional pharmaceutically acceptable excipients and/or vehicles.

For internal administration the α-interferon may, for example, be formulated in conventional manner for oral or rectal administration. Formulations for oral administration include aqueous solutions, syrups, elixirs, powders, granules, tablets and capsules which typically contain conventional excipients such as binding agents, fillers, lubricants, disintegrants, wetting agents, suspending agents, emulsifying agents, preservatives, buffer salts, flavoring, coloring and/or sweetening agents.

The α-interferon component of the therapeutic procedures is preferably Alferon N Injection® the only approved natural, multi-species, α-interferon available in the United States. It is the first natural source, multi-species interferon and is a consistent mixture of at least seven species of α-interferon. In contrast, the other available α-interferons are single molecular species of α-interferon made in bacteria using DNA recombinant technology. These single molecular species of α-interferon also lack an important structural carbohydrate component because this glycosylation step is not performed during the bacterial process.

Unlike species of α-interferon produced by recombinant techniques, Alferon N Injection® is produced by human white blood cells which are able to glycosylate the multiple α-interferon species. Reverse Phase HPLC studies show that Alferon N Injection® is a consistent mixture of at least seven species of alpha interferon (α2, α4, α7, α8, α10, α16, α17). This natural-source interferon has unique anti-viral properties distinguishing it from genetically engineered interferons. The high purity of Alferon N Injection® and its advantage as a natural mixture of seven interferon species, some of which, like species 8b, have greater antiviral activities than other species, for example, species 2b, which is the only component of Intron A. The superior antiviral activities for example in the treatment of chronic hepatitis C virus (HCV) and (HIV) and tolerability of Alferon N Injection® compared to other available recombinant interferons, such as Intron A and Roferon A, have been reported.

It is reported Alferon N Injection® has activity against a natural coronavirus infection in pigs. Transmissible gastroenteritis (TGE) coronavirus causes an acute gastroenteritis in swine. The diarrhea and dehydration caused by this viral infection result in a high mortality rate in neonates with severity inversely related to the age of the animal. In fact, in piglets less than 14 days of age the morality/morbidity rate typically approaches 100%. Piglets, ages 1-12 days treated with 1.0, 10.0, or 20.0 IU of Alferon N Injection® were found to have an increased survival compared to the control group indicating benefit of this natural mixture of α-interferons in combating this particular coronavirus.

The invention includes methods of enhancing therapy against coronaviruses or a susceptible viral infection other than coronaviruses which has a common mechanism of viral multiplication or pathogenesis, in whole or in part, similar to that of coronaviruses by administering to patients interferons, particularly natural human alpha interferon and together or conjointly a synthetic, specifically configured, double-stranded ribonucleic acid (dsRNA). The dsRNA of choice is Ampligen®, a synthetic, specifically configured, double-stranded ribonucleic acid (dsRNA) which retains the immunostimulatory and antiviral properties of other double-stranded RNA molecules (dsRNA) but exhibits greatly reduced toxicity. Like other dsRNA, Ampligen® can elicit the induction of interferon and other cytokines. Ampligen® has the ability to stimulate a variety of dsRNA-dependent intracellular antiviral defense mechanisms including the 2′, 5′-oligoadenylate synthetase/RNase L and protein kinase enzyme pathways.

The mismatched dsRNA may be of the general formula rIₙ·r(C₁₂U)ₙ. In this and the other formulae that follow r=ribo. Other mismatched dsRNAs for use in the present invention are based on copolynucleotides selected from poly (Cₘ,U) and poly (CₘG) in which m is an integer having a value of from 4 to 29 and are mismatched analogs of complexes of polyriboinosinic and polyribocytidilic acids, formed by modifying rIₙ·rCₙ to incorporate unpaired bases (uracil or guanine) along the polyribocytidylate (rCₘ) strand. Alternatively, the dsRNA may be derived from r(I)·r(C) dsRNA by modifying the ribosyl backbone of polyriboinosinic acid (rIₙ), e.g., by including 2′-O-methyl ribosyl residues. The mismatched may be complexed with an RNA-stabilizing polymer such as lysine cellulose. Of these mismatched analogs of rIₙ·rCₙ, the preferred ones are of the general formula rIₙ·r(C₁₁₋₁₄,U)_{n.} or rIₙ·r(C₂₉,G)ₙ, and are described by Carter and Ts'o in U.S. Pat. Nos. 4,130,641 and 4,024,222 the disclosures of which are hereby incorporated by reference. The dsRNA's described therein generally are suitable for use according to the present invention.

Other examples of mismatched dsRNA for use in the invention include:
r(I)·r(C₄, U)
r(I)·r(C₇, U)
r(I)·r(C₁₃, U)
r(I)·r(C₂₂, U)
r(I)·r(C₂₀, G) and
r(I)·r(C_{p·23},G_{>p}).

Alternatively the dsRNA may be the matched form, thus polyadenylic acid complexed with polyuridylic acid (poly A·poly U) may also be used.

When administered 24 hours prior to viral challenge, ampligen has been demonstrated in viral cytopathic inhibition assays and neutral red assays to inhibit human coronavirus strain OC-43, thus suggesting protective activity of ampligen against human chromavirus prior to an encounter with this virus.

α-interferon and/or the dsRNA may be administered for therapy by any suitable route including oral, rectal, nasal, topical (including transdermal, buccal and sublingual), vaginal and parenteral (including subcutaneous, intramuscular, intravenous intradermal, and intravitreal). It will be appreciated that the preferred route will vary with the condition and age of the recipient, the nature of the infection and the chosen active ingredient.

As indicated above, severe acute respiratory syndrome (SARS) is caused by a newly identified member of the coronavirus family. Ampligen®, a double-stranded RNA (dsRNA), is reported to exhibit antiviral activity against the coronavirus, Mouse Hepatitis Virus Type-3 (MHV-3) see Hepatology 3:837, 1983. MHV-3 is a coronavirus which causes both a fulminant and a chronic form of hepatitis depending on the mouse strain studied. Ampligen® treatment had a positive effect against the MHV-3 coronavirus in both mouse models. In an acute infection model, Balb/cJ mice exposed to MHV-3 and then treated twice with Ampligen® survived up to four times longer than untreated mice. Since no treatment was given beyond 24 hours post-exposure, it is likely that additional Ampligen® treatments would have had an even greater impact on survival. In the chronic hepatitis model, C3H mice treated after exposure to the MHV-3 coronavirus cleared the virus quickly and did not develop chronic hepatitis. Thus, Ampligen® has shown activity against the coronavirus, MHV-3, in two different mouse models, increasing survival in the acute infection model and completely abrogating the infection in the chronic model.

In a further study it has been determined Alferon® inhibits SARS-CoV at a high specific activity in Vero 76 cells (African green monkey) in culture. Alferon® is a highly purified natural α-interferon obtained from human leucocytes and consists of seven different α-interferon amino acid sequences (α2, α4, α7, α8, α10, α16, and α17). Inhibition was quantitated by visual cytopathic effect (CPE), inhibition of the cellular uptake of the vital dye, neutral red (NR), and by virus yield reduction. NR assay was conducted immediately following visual assay. Fifty-percent effective doses (EC5O) were calculated for both CPE and NR assays by regression analysis. Quantitative values for viral yield reduction assays were expressed as 90% effective concentrations (EC9O), representing the drug concentration required to reduce SARS-CoV titers by one log₁₀ and were calculated by regression analysis. Vein cells infected at a MOI of 0.001 visually exhibited 100% CPE over a 3-5 day incubation period without treatment. Alferon® inhibited SARS-CoV at an ECSO=5,696±1,703 (SEM) IU/ml (visual) and 10,740±5,161 (SEM) IU/ml (NR). Viral load reduction by one log₁₀ was 78,000±22,000 (SEM) IU/ml.

The beneficial effects of Alferon® are also reported by Tan et al, Emerging Infectious Diseases—www.cdc.gov/eid—Vol. 10 No. 4, April 2004, in which several commercially available, clinically approved compounds from several antiviral pharmacologic classes screened to determine the presence of in vitro anti-SARS-CoV activity. Of the 18 antiviral compounds tested Alferon® was found to be the most active FDA-approved interferon when tested against the SARS coronavirus. This contrasts with the recombinant α-interferons, Roferon and Intron A, which were not found to be active against the SARS coronavirus when tested at much higher concentrations.

It is also reported the activity of interferon can potentially be amplified by the addition of a double-stranded RNA drug, Ampligen®. While interferon up-regulates certain intracellular antiviral pathways, dsRNAs, like Ampligen®, are required to fully activate these important antiviral pathways. When interferons are combine with Ampligen® synergistic antiviral and antitumor effects are seen. Moreover, Ampligen® has already shown strong antiviral activity in two separate animal models of the coronavirus (MHV-3). Although uncertainty now exists regarding the characteristics of this coronavirus, in the event it is determined the SARS-associated coronavirus elaborates IFN neutralizing products, Ampligen® has potential to override these inhibitors and achieve an antiviral effect.

In addition, as a further attribute of the dsRNA arm of the disclosed therapeutic combination therapy, synergistic antiviral and antitumor effects have been demonstrated using Ampligen® treatment in combination with all three types of interferon (α, β and γ). These synergistic effects have been seen against HIV and a variety of different histologic tumor types. Four human tumor cell lines were studied for their response to antiproliferative effects of Ampligen® in combination with various interferons. Results indicate that (1) Ampligen® worked synergistically with all interferons in all cell lines studied; (2) growth inhibition of cells resistant to interferons can be potentiated by low doses of Ampligen®; (3) the antiproliferative effect of interferons can be potentiated by Ampligen® in Ampligen®-resistant cells; and (4) Ampligen® works by a mechanism(s) other than, or in addition to, the induction of interferon. See Montefiori, AIDS Res. and Human Retroviruses 5:193-203, 1989 and Hubbell, Int. J. Cancer 37:359-365, 1986.

The recommended dosage of the components will depend on the clinical status of the patient and the experience of the clinician in treating similar infection. As a general guideline dosage of Alferon N Injection® utilized for systemic infections is 5 to 10 million units (sq) thrice weekly. Experience to date is with dosages above 3 IU/lb of patient body weight. Oral α-interferon (Alferon LDO) has been administered as a liquid solution in the range of 500-2000 IU/day and calculated on the basis of a 150 pound human this is 3.3 to 13.3 IU/lb per day.

Our experience indicates beneficial results are obtained at dosage levels of α-interferon in excess of 450 IU, that is greater than 3 IU/pound body weight. These amounts are in contrast to and greater than Cummins use in U.S. Pat. No. 5,910,304 of alpha interferon administration to the pharyngeal mucosa orally or as lozenge or tablet.

The Ampligen® dose schedule is 400 mg by IV infusion twice weekly, although these amounts and/or dosage frequency may be varied by the clinician in response to the patient's condition. The components may be administered at the same time, for instance as mixture of the α-interferon and dsRNA, independently as the α-interferon then the dsRNA or the α-interferon and the dsRNA may be administered in a time-spaced manner.

Another aspect of the invention is the treatment of acute and chronic viral infections including, for example, HIV, severe acute respiratory syndrome and avian influenza employing a synergistic combination of an α-interferon such as Alferon with another antiviral such as ribavirin and, optionally, also, with a dsRNA.

Interferons, particularly α-interferons and dsRNAs are discussed above.

Synergistic activity of α-interferon and ribavirin has been demonstrated to inhibit an avian virus (Duck/MN/1525/81) in cell culture as illustrated in Example 2. The particular cells used in this study lack receptors for dsRNAs, however based upon our experience it is expected that cells with receptors for dsRNAs will also exhibit beneficial even synergistic effects of a tri-component administration of an α-interferon, an antiviral such as ribavirin, and a dsRNA such as Ampligen®. It is expected this 3-part combination will be effective in treating various acute or chronic viral infections such as HIV and severe acute respiratory syndrome as well as avian influenza.

A further aspect of the invention is conferring resistance to, or the treatment of or mitigation of the effects influenza viruses generally and avian flu virus in particular or a susceptible viral infection other than avian flu which has a common mechanism of viral multiplication or pathogenesis, in whole or in part, similar to that of avian flu using a combination of (1) an antiviral agent that inhibits influenza virus neuraminidase and possibly altering of virus particle aggregation and release, and (2) a dsRNA as described above.

As an example of antiviral agent (1) oseltamivir or its pharmaceutically acceptable salts such as the carboxylate or the phosphate which is available as TAMIFLU® from Roche as 75mg capsules for oral use. TAMIFLU is used for postexposure and preseasonal prophylaxis of influenza infections.

### EXAMPLE 1

Effects of Alferon N®, an alfa-n3 human interferon, on the replication of SARSCoV in vitro.

Vero 76 cells (African green monkey kidney) were obtained from American Type Culture Collection (Manassas, Va.). The growth medium was Eagle's minimum essential medium with non-essential amino acids (MEM), 5% FBS and 0.1% NaHCO₃. The test medium was MEM supplemented with 2% FBS, 0.18% NaHCO₃ and 50 μg gentamicin/ml. The SARS coronavirus, strain 200300592 (Urbani), was obtained from James Comer (Centers for Disease Control, Atlanta, Ga.). Human leukocyte derived interferon alfa-n3 (03-6600) was kindly provided by Hemispherx Biopharma, Inc. (New Brunswick, N.J.) as a stock solution of 5×10⁶ units/ml.

Using cytopathic effect (CPE) reduction assays read visually and verified spectrophotometrically by neutral red (NR) uptake assay of the same plate (Barnard et al., 2001), an interferon alfa-n3 was evaluated for anti-SARSCoV activity in Vera 76 cells.

Virus at a multiplicity of infection of 0.001 was added to 96 well plates seeded with near confluent monolayers of cells in which drug had been serially diluted, using 10-fold or ½ log dilution series. Addition of virus was within five minutes after exposure of cells to drug.

The cells were incubated at 37° C. until the untreated virus controls displayed destruction of the monolayers (100% CPE, 3-5 days). The plates were then scored for cytotoxicity and viral CPE by microscopic examination, usually followed immediately by neutral red staining and processing for spectrophotometric reading. EC50 values (the concentration of compound needed to inhibit the cytopathic effect to 50% of the control value) and IC50 values (the concentration at which uptake of neutral red or cytotoxic effects was reduced by 50% compared to control cells) was calculated by regression analysis. Values were expressed as mean ± the standard error of the mean. For the visual assay, n=10 and for the NR assay, n=5. A selective index (IC50/EC5O) for each compound was then calculated. Compounds found active by these assays were then further evaluated for inhibitory activity in two separate virus yield reduction assays (Barnard et al., 2001). EC9O values were derived by regression analysis from those assays and represent the concentration at which virus yields were reduced by 1 log₁₀. The EC9O values were averaged and the average was expressed as the mean the ± standard error of the mean.

It was found Alferon N® inhibited SARSCoV, with an EC5O=5,696±1703 IU by visual CPE inhibition assay and an EC5O 10,740±5,161 IU/ml by NR assay. However, viral cytopathic effects (CPE) were readily apparent at all dilutions tested, although at higher compound doses, CPE was greatly reduced compared to the virus replication controls (data not shown). This phenomenon was verified by virus yield reduction assay in which each dilution of drug was sampled and quantified for the presence of surviving virus or newly produced virus. At each dilution of compound, infectious virus was detected, with lower amounts of virus being detected at the higher concentrations of compound (data not shown). The concentration at which virus load was reduced by 1 log 10 (EC9O) 78 000±22,000 IU/ml.

From these studies it was determined Alferon N® worked well in reducing virus cytopathic effect, with an EC5O of 5,696±1703 IU/ml.

### EXAMPLE 2

Tables 1 and 2 show the results of two experiments testing Alferon N in combination with ribavirin against an influenza A (H5N1) virus in Vero cells. The testing procedure involved an 18-hour pre-incubation of compounds (half-log increments) with the cells prior to virus exposure in order to activate them to an interferon-induced state. Fresh Alferon N, ribavirin, or the both used together was added again at the time of infection. After three days of incubation of virus compounds, visual examination was used to assess extent of cell destruction (cytopathic effect (CPE)) caused by the virus.

Tables 1 and 2 show actual percentages of cell destruction observed at each drug combination. The cytopathic effect (CPE) inhibition assay results indicated that Alferon N alone had a weak effect at 1,000, 3,200 and 10,000 units/ml. The combination of Alferon N plus ribavirin (32 ug/ml) showed CPE suppression that was greater than either drug alone. Ribavirin at 100 ug/ml showed a 25-50% CPE. Less CPE (i.e. protection against the virus) was observed with ribavirin (100 ug/ml) plus Alferon N (four doses).

The overall assessment is that there was improvement in cell protection when Alferon N was combined with ribavirin.

Table 1. Effect of combination of Alferon N and ribavirin on an Influenza A/Duck/MN/1525/81 (H5N1) infection in Vero cells as determined by cytopathic effect inhibition assay.

TABLE 1
Percent Cytopathic Effect

| | |
|---|---|
| Alferon N | Ribavirin (μg/ml) |

| (Units/ml) | 320 | 100 | 32 | 0 |
|---|---|---|---|---|
| 10,000 | 0 | 0 | 13 | 75 |
| 3,200 | 0 | 0 | 25 | 80 |
| 1,000 | 0 | 0 | 25 | 88 |
| 320 | 0 | 13 | 25 | 100 |
| 0 | 0 | 25 | 100 | 100 |

Table 2. Experiment 2. Effect of combination of Alferon N and ribavirin on an Influenza A/Duck/MN/1525/81 (H5N1) infection in Vero cells as determined by cytopathic effect inhibition assay.

TABLE 2
Percent Cytopathic Effect

| | |
|---|---|
| Alferon N | Ribavirin (μg/ml) |

| (Units/ml) | 320 | 100 | 32 | 0 |
|---|---|---|---|---|
| 10,000 | 0 | 0 | 50 | 71 |
| 3,200 | 0 | 13 | 67 | 79 |
| 1,000 | 0 | 25 | 88 | 100 |
| 320 | 0 | 25 | 100 | 100 |
| 0 | 25 | 50 | 100 | 100 |

Cinatl J, Morgenstern B, Bauer G, Chandra P, Rabenau H, Doerr H W. Treatment of SARS with human interferons. Lancet 2003; 362: 293-4.

Barnard D L, Stowell V D, Seley K L, Hegde V R, Das S R, Rajappan V P, et al. Inhibition of measles virus replication by 5′-nor carbocyclic adenosine analogues. Antiviral Chem Chemother 2001; 12: 241-250.

### EXAMPLE 3

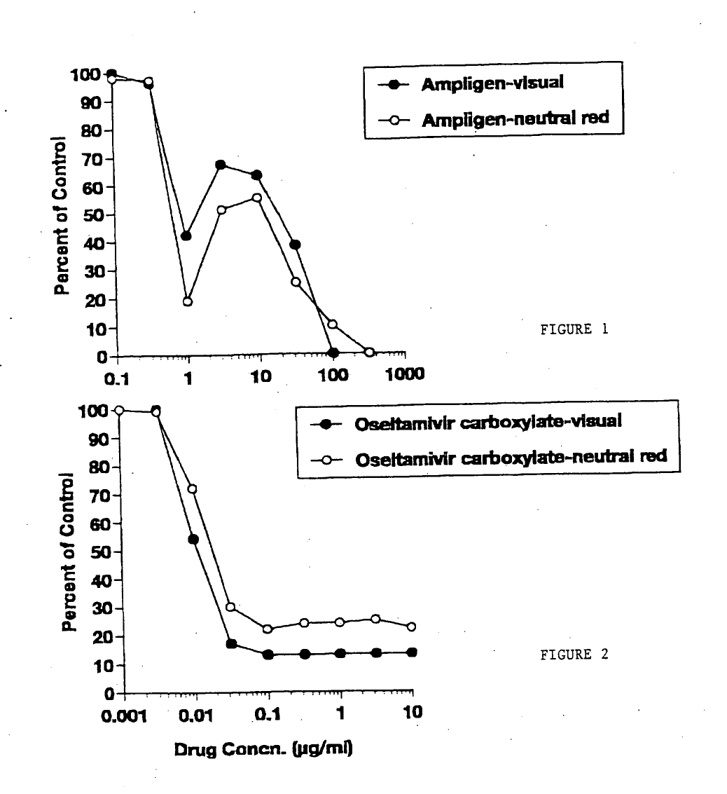

The influence of Ampligen® and oseltamivir carboxylate on influenza virus was studied to determine the cytopathic effect of these two products, individually. Ampligen® and oseltamivir carboxylate were tested against an influenza A/Duck/MN/1525/81 virus in MDCK (dog cell) cell culture. The compounds were pre-incubated with cells 18 hours prior to addition of virus. Fresh compound was applied at the time of virus addition. As shown in FIG. 1, a zone of activity was observed for Ampligen® treatment between 1 and 32 μg/ml where some microwells were protected and others were not. As shown in FIG. 2, oseltamivir carboxylate was active down to 0.032 μg/ml.

### EXAMPLE 4

In this example Ampligen® was examined in combination with oseltamivir carboxylate (Tamiflu) for effectiveness against an influenza (avian flu) A/Duck/MN/1525/81 (H5N1) virus infection in MDCK cells. The testing procedure involved an 18-hour pre-incubation of the compounds (half-log increments) with the cells prior to virus exposure in order to activate them to an interferon-induced state. Fresh medium containing Ampligen®, oseltamivir carboxylate, or the both used together was added again at the time of infection. After three days of incubation of virus with compounds, visual and neutral red straining procedures were used to assess extent of cell destruction caused by the virus.

The data obtained and reported in the following Table 3 shows percentages of cell destruction observed at each drug combination. The cytopathic effect inhibition (CPE) assay results indicated that Ampligen® alone had a good effect only at 100 μg/ml. The combination of Ampligen® at lower concentrations plus oseltamivir carboxylate showed CPE suppression that was greater than either drug alone at several combinations.

The following table shows the effect of a combination of Ampligen® and oseltamivir carboxylate on an Influenza A/Duck/MN/1525/81 (H5N1) infection in MDCK cells as determined by cytopathic effect inhibition assay.

TABLE 3
Percent of Cytopathic Effect

| Ampligen ® | Oseltamivir Carboxylate (μg/ml) | | | | | |
|---|---|---|---|---|---|---|
| (μg/ml) | 0.32 | 0.1 | 0.032 | 0.01 | 0.0032 | 0 |
| 100 | 0 | 0 | 0 | 0 | 0 | 0 |
| 32 | 0 | 0 | 0 | 0 | 0 | 75 |
| 10 | 0 | 0 | 0 | 58 | 63 | 100 |
| 3.2 | 13 | 13 | 29 | 100 | 71 | 100 |
| 1.0 | 13 | 13 | 50 | 100 | 100 | 100 |
| 0.32 | 13 | 13 | 50 | 100 | 100 | 100 |
| 0 | 25 | 54 | 100 | 100 | 100 | 100 |

The data was analyzed to determine a Combination Index (CI) and is based on the multiple drug-effect equation of Chou-Talalay (Chou, T.-C. and Talalay, P., J. Biol. Chem. 252: 6438-6442, 1977) in which CI values less than 1.0 indicate synergism. The results are presented in Table 4:

TABLE 4
CI for Experimental Values

| Ampligen ® | Ose_Car | |
|---|---|---|
| (μg/ml) | (μg/ml) | CI |
| 0.32 | 0.32 | 0.723 |
| 0.32 | 0.1 | 0.228 |
| 0.32 | 0.032 | 0.378 |
| 0.32 | 0.0032 | 0.160 |
| 1 | 0.032 | 0.287 |
| 1 | 0.0032 | 0.136 |
| 3.2 | 0.032 | 0.231 |
| 3.2 | 0.01 | 0.765 |
| 3.2 | 0.0032 | 0.131 |
| 10 | 0.01 | 0.312 |
| 10 | 0.0032 | 0.287 |

All combinations analyzed showed synergism indicating a clear advantage for the combination which provided an improvement in cell protection when Ampligen® was combined with and enhances oseltamivir carboxylate in inhibiting cell destruction by avian influenza.

It is expected that the combined use of Ampligen® as a prototypic dsRNA and oseltamivir as a prototypic neuraminidase inhibitor will be effective in treating various acute and chronic viral infections such as influenza, severe acute respiratory syndrome as well as avian influenza. The discovery of the combination of a neuraminidase inhibitor and dsRNA giving synergistic antiviral inhibition is an unexpected finding with great promise to mitigate the emerging problems in terms of controlling the expected pandemic with neuraminidase inhibitors given alone.

What is claimed is:

1. A method of treating severe acute respiratory syndrome comprising administering to an infected subject natural human alpha interferon.

2. A method of treating severe acute respiratory syndrome comprising administering to an infected subject a dsRNA.

3. A method of treating severe acute respiratory syndrome comprising the coordinated administration to an infected subject of (1) a natural human alpha interferon and (2) dsRNA.

4. The method of claim 2 or 3 wherein the dsRNA is rIₙ·r(C₁₂U)_{n.}, Poly A·Poly U or rIₙ·r(C₂₉,G)ₙ, in which r is ribo.

5. The method of claim 1 wherein the interferon is administered orally, nasally, IV, IM or SQ in an amount of at least 3 IU per pound of the subject's body weight.

6. A method of mitigating the effects of or conferring resistance to severe acute respiratory syndrome or a susceptible viral infection other than severe acute respiratory syndrome which has a common mechanism of viral multiplication or pathogenesis, in whole or in part, similar to that of severe acute respiratory syndrome comprising, prior to exposure, to the SARS-CoV or shortly after exposure to the SARS-CoV, but prior to the development of symptoms, administering to a subject natural human α-interferon.

7. The method of claim 6 wherein the interferon is administered orally, nasally, IV, IM or SQ.

8. A method of treating avian influenza or a susceptible viral infection other than avian flu which has a common mechanism of viral multiplication or pathogenesis, in whole or in part, similar to that of avian flu comprising administering to an infected subject natural human alpha interferon and ribavirin, and/or neuraminidase inhibitors.

9. A method of treating avian influenza comprising the coordinated administration to an infected subject of (1) a natural human alpha interferon, (2) ribavirin, (3) dsRNA, and (4) a neuraminidase inhibitor.

10. The method of claim 8 or 9 wherein the dsRNA is rIₙ·r(C₁₂U)_{n.}, Poly A·Poly U or rIₙ·r(C₂₉,G)ₙ, in which r is ribo.

11. The method of claim 8 or 9 wherein the interferon is administered orally, nasally, IV, IM or SQ in an amount of at least 3 IU per pound of the subject's body weight.

12. A method of mitigating the effects of or conferring resistance to avian influenza or a susceptible viral infection other than avian influenza which has a common mechanism of viral multiplication or pathogenesis, in whole or in part, similar to that of avian influenza comprising, prior to exposure, to the avian influenza or shortly after exposure to the avian influenza, but prior to the development of symptoms, administering to a subject (1) natural human α-interferon in combination with (2) ribavirin, or other chemotherapeutics such as newer neuraminidase inhibitors.

13. A method of mitigating the effects of or conferring resistance to avian influenza or a susceptible viral infection other than avian influenza which has a common mechanism of viral multiplication or pathogenesis, in whole or in part, similar to that of avian influenza comprising, prior to exposure, to the avian influenza or shortly after exposure to the avian influenza, but prior to the development of symptoms, administering to a subject (1) natural human α-interferon in combination with (2) ribavirin and (3) a dsRNA.

14. The method of claim 12 or 13 wherein the interferon is administered orally, nasally, IV, IM or SQ in an amount of at least 3 IU per pound of the subject's body weight.

15. A method of mitigating the effects of or conferring resistance to influenza comprising, prior to exposure, to the influenza or shortly after exposure to the influenza, but prior to the development of symptoms, administering to a subject (1) a dsRNA in combination with (2) a neuraminidase influenza virus inhibitor.

16. A method of mitigating the effects of or conferring resistance to avian influenza or a susceptible viral infection other than avian influenza which has a common mechanism of viral multiplication or pathogenesis, in whole or in part, similar to that of avian influenza comprising, prior to exposure, to the avian influenza or shortly after exposure to the avian influenza, but prior to the development of symptoms, administering to a subject (1) a dsRNA in combination with (2) a neuraminidase influenza virus inhibitor.

17. The method of claim 15 or 16 wherein the dsRNA is rIₙ·r(Cl₂U)_{n.}, Poly A·Poly U or rIₙ·r(C₂₉,G)ₙ, in which r is ribo.

18. The method of claim 15 or 16 wherein the neuraminidase inhibitor is oseltamivir, zanamivir, amantadine, rimantadine or a pharmaceutically acceptable salt thereof.
